(19) United States
(12) Patent Application Publication
(10) Pub. No.: US 2018/0085595 A1
(43) Pub. Date: Mar. 29, 2018

(54) METHODS FOR INDUCING NEUROGENESIS

(71) Applicant: THE LITEBOOK COMPANY LTD., Medicine Hat (CA)

(72) Inventors: Robert James SUTHERLAND, Lethbridge (CA); Terry M. COOK, Kelowna (CA)

(21) Appl. No.: 15/716,224

(22) Filed: Sep. 26, 2017

Related U.S. Application Data
(60) Provisional application No. 62/399,817, filed on Sep. 26, 2016.

(51) Int. Cl. A61N 5/06

(52) U.S. Cl. CPC A61N 5/0622; G01N 33/5058; A61N 5/0618

(57) ABSTRACT

A method for inducing neurogenesis in the brain of a mammal includes administering a light treatment from an LED light source.

### CROSS REFERENCE TO RELATED APPLICATION

This application claims priority to U.S. Provisional Application 62/399,817 filed Sep. 26, 2016, the disclosure of these prior applications are hereby incorporated in their entirety by reference.

### FIELD

The present description relates to methods for neurogenesis.

### BACKGROUND

Neuronal loss is an undesired pathological condition of aging. Illness such as stroke or Alzheimer's disease, traumatic brain injury and depression can also cause neuronal loss and lead to cognitive decline.

The stimulation of neurogenesis may be useful in treating neuronal loss and may lead to maintained or improved cognitive functions.

Normal treatments to induce neurogenesis include administration of pharmaceuticals.

### SUMMARY

In accordance with one aspect of the present invention, there is provided a method for inducing neurogenesis in a brain, the method comprising: administering to a mammal a light therapy treatment including light from a light emitting diode (LED) light source.

In accordance with another aspect of the present invention, there is provided a method for treating a mammal afflicted with a neurodegenerative disease or condition, comprising administering a light treatment including light from an LED light source to induce neurogenesis.

In accordance with another broad aspect, there is provided, a use of a light therapy device according to one of the embodiments described herein, for inducing neurogenesis in the brain.

### BRIEF DESCRIPTION OF THE DRAWINGS

FIG. 1 is a front elevation view of a light therapy device according to the present invention. A portion of the device has been cut away to facilitate illustration of internal components.

FIG. 2 is a side elevation view of the light therapy device of FIG. 1 with the support leg folded against the housing.

FIG. 3 is a sectional view along line A-A of FIG. 1.

FIG. 4 is a graph showing a spectra analysis of light emitted by one embodiment of a light therapy device.

FIG. 5 is a schematic view of a method of light therapy.

FIG. 6 is another schematic view of a method of light therapy.

FIG. 7 is a graph showing the number of BrdU positive cells in the hippocampus of a rat after treatment with light (LT) and no light (noLT) groups as compared to control.

FIG. 8 is a graph showing the number of Ki67 positive cells in the hippocampus of a rat after treatment with light (LT) and no light (noLT) groups as compared to control.

FIG. 9 is a graph showing the number of DCX positive cells in the hippocampus of a rat after treatment with light (LT) and no light (noLT) groups as compared to control.

FIG. 10 is a graph showing the number of Ki67 positive cells in the hippocampus of a rat after treatment with light alone, exercise alone, and the combination of light and exercise as compared to control.

FIG. 11 is a graph showing the number of BrdU positive cells in the hippocampus of a rat after treatment with light alone, exercise alone, and the combination of light and exercise as compared to control.

FIG. 12 is a graph showing the number of DCX positive cells in the hippocampus of a rat after treatment with light alone, exercise alone, and the combination of light and exercise as compared to control.

### DETAILED DESCRIPTION

It is to be understood that the following description is not to be taken as limiting the invention. It is to be understood that the terminology used herein is used for the purpose of describing embodiments only and is not intended to limit the scope of the present invention. Unless otherwise defined, the technical and scientific terms used herein have the same meaning as commonly understood by one of ordinary skill in the art to which the invention belongs.

The invention offers methods and uses for enhancing neurogenesis in the brain. In one embodiment, the applicants have determined that ocular light treatment with light from an LED light source affects the formation of new brain cells in the cerebral cortex of a mammal. Applicants have found that ocular light treatment from an LED light source induced hippocampal neurogenesis. These results were found to occur in natural subjects, in particular mammals that had not been genetically altered.

In some experiments rats experienced significant daily aerobic exercise in running wheels and they had experienced significant chronic circadian disruption prior to initiating light treatment. It has been repeatedly shown in the literature that exercise alone can have some positive effects on both remembering and neurogenesis. In addition, stress associated with circadian disruption could have significantly suppressed the baseline of adult neurogenesis prior to the onset of light treatment. Thus, further experiments were conducted to show the impact of light therapy alone on adult hippocampal neurogenesis in animals with and without significant amounts of daily exercise, without genetic transfection and without any chronic circadian disruption.

Applicants have determined that ocular light treatment can enhance adult hippocampal neurogenesis in mammals.

These results represent encouraging outcomes with direct implications for benefit in relevant human conditions and for possible brain mechanisms that directly influence age-related and other cognitive processes.

The terms “treatment,” “treating,” “treat,” “therapy,” “therapeutic,” and the like are used herein to refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease.

The light therapy found to be effective is from an LED light source. In one embodiment, the LED generated light has a peak in the blue to green wavelengths of 400 to 600 nm and in one embodiment 450 to 550 nm. The light useful for light therapy can include a spectrum of wavelengths with a maximum peak in the range of 400 to 600 nm. Such light may, for example, appear as white light. While there may be more than one peak wavelength in the emitted light, the major peaks are preferably in the 400 to 600 nm range. In one embodiment, a maximum peak wavelength can in the blue region of the spectrum, which is 420 to 505 nm. In one embodiment, a maximum peak wavelength can be in the range of 446 to 477 nm.

The useful intensity of the light is bright and may be in the range of 500 to 12,000 lux at 12 inches. In one embodiment, the light has an intensity of between 500 and 2,500 lux at 12 inches.

A device for emitting LED-sourced white light may include a light emitting assembly including a plurality of LEDs capable of generating a spectrum of light appearing to be white and at an intensity of 500 to 12,000 lux at 12 inches.

Such light sources are available from various sources and in various configurations including hand held units, clinical units, wearable units, or product integrated units such as computer monitor integrated units. For example, small hand held light therapy devices are available from The LED light Company Ltd., Alberta, Canada. For example, LED light™ products including LED lights and known as LED light Advantage™ and LED light Edge™ emit about 10,000 lux at 20 to 24 inches and the Litebook Elite™ emits about 2,500 lux at 12 inches.

LED light emitting devices suitable for use to induce neurogenesis can be small and lightweight. Suitable LED devices can be hand held: being portable and lightweight while also being durable and energy efficient. The device may be useful in confined spaces, during travel and for in-flight use while being aesthetically acceptable.

An ocular light treatment device can include an outer housing including an opening; a light emitting assembly in the housing and operable to emit light through the opening in the housing, the light emitting assembly including a plurality of LEDs capable of generating less than 12,000 lux at 12 inches.

The housing can be formed to permit the device to be mounted on a support surface or stand in a spaced relation from a user. For example, the housing can include a support base on which the device can be set on a support surface, the housing can include a support leg for supporting the device in an upright configuration and/or the housing can include an electrical contact for electrical connection to a mounting device. The support base, if one is included, can be formed in a flatted configuration and or can be weighted relative to the remainder of the housing to permit setting the device in an upright configuration. Alternately or in addition, the support base can be formed to be engaged by a holder for supporting the housing on a support surface in an upright configuration. The device can be generally intended to be operated at a distance of about 12 or more inches from the user and positioned with the opening toward the user's eyes so that the light emitted therefrom can pass directly or indirectly to the user's eyes.

The LEDs can provide a light emitting assembly that can be light-weight and durable. In one embodiment, the LEDs can be arranged in a pattern over an area and the light emitting assembly can be selected to emit light from the LEDs directly towards the user's eyes.

The light emitting assembly can include a screen of transparent or translucent material positioned over the LEDs, for example, across the opening to seal the housing and to prevent access to the LEDs and other internal components. The screen can be formed of light diffusing sheet material to provide a more uniform emission of light and/or to adjust the lux or characteristics of the light. While LEDs do not emit any significant amount of ultraviolet radiation, the diffuser sheet material can include a UV filter, if desired.

The LEDs can be selected to emit light illuminances of up to 12,000 lux and in one embodiment less than 10,000 lux and possibly even less than 2,500 lux (all measured at 12 inches from the assembly). The light levels can be selected in this range to be effective using reasonable treatment durations, but can reduce visual glare and other side effects and to simplify the device such as by reducing the number or power of LEDs and, accordingly, the size, cost and weight of the device. Lower light levels can also reduce device power requirements, therefore, facilitating the use of battery power.

The light emitted by the light emitting assembly either as emitted by the LEDs or as adjusted by a screen over the LEDs, can be selected to have a peak in the blue to green wavelengths of 400 to 600 nm and in one embodiment 450 to 550 nm. The emitted light can be exclusively in the blue to green wavelengths such that it visually appears blue to green. Alternately, the light emitted can include a spectrum of wavelengths with a maximum peak in the range of 400 to 600 nm. Such light may, for example, appear as white light. While there may be more than one peak wavelength in the emitted light, the major peaks are preferably in the 400 to 600 nm range. In one embodiment, a maximum peak wavelength can in the blue region of the spectrum, which is 420 to 505 nm. In one embodiment, a maximum peak wavelength can be in the range of 446 to 477 nm.

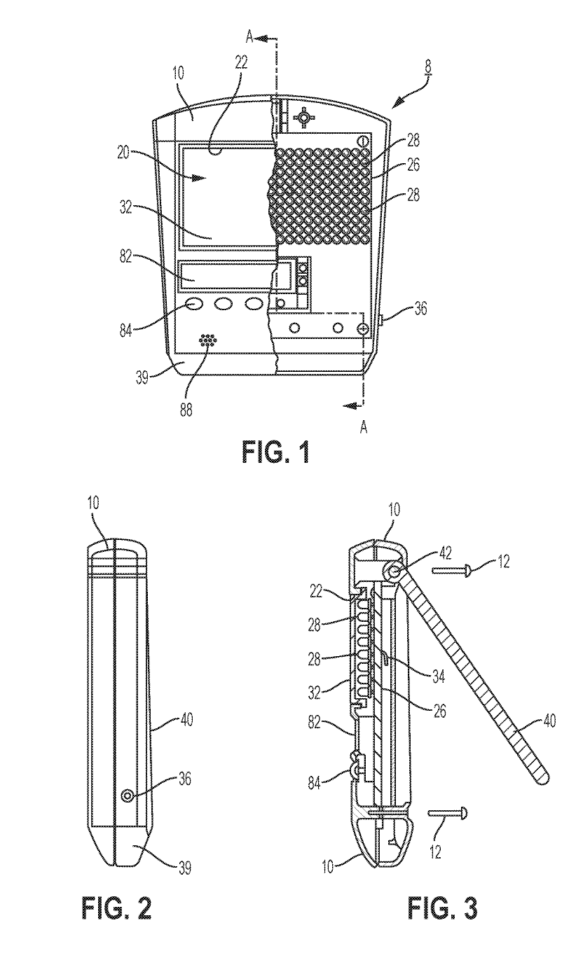

Referring to FIGS. 1 to 3, a light therapy device 8 according to one embodiment is shown. The device can be small in size, for example, resembling a large calculator or hand-held computer. The outside dimensions of the device can be less than about 7 inches×7 inches×1.5 inches. The size can be varied as desired and with consideration as to portability, convenience and the components that must be contained within the device.

The device can include an outer housing 10. The housing can be formed of a durable, impact resistant material such as, for example, a polymer (i.e. nylon, thermoplastics or blends thereof). All housing parts can be of minimal thickness to provide suitable impact resistance and support for internal components while minimizing the weight of the device. The housing can be formed in various ways, for example, from injection molded parts secured together by screws 12 or other fasteners, polymeric welding, fusing, adhesives, etc.

The housing can carry a light emitting assembly 20. The light emitting assembly can be mounted in the housing such that, during operation, light emitted therefrom is directed out through an opening 22 in the housing. The light can be emitted in a broad, as opposed to a focused, beam. The broad beam can increase in its width with increasing distance from the device so that light impinging on the user is about shoulder width (30 to 50 inches). For example, in one embodiment, light can be emitted from the device at an angle of about 10° to 30° from an axis oriented orthogonally through the plane of the opening. In one embodiment, the light emitting assembly can generate a beam of light that radiates out through the opening having a width of about 4.5″ to a beam width of about 40″ at 24″ from the device. This then can create a treatment field of about shoulder width when the device is operated at 24″ from the user.

Light emitting assembly 20 can include a printed circuit (PC) board 26 providing electrical connection for light emitting diodes 28. The LEDs can be mounted in various ways, for example as by traditional mounting or surface mounting. A screen 32 can be mounted over the light emitting diodes and across the housing opening to prevent access to the internal components of the device. If a screen is used, it is useful to ensure that appropriate light characteristics, as set out herein, can passed therethrough to permit treatment. In one embodiment, screen 32 includes quesnel lens configurations.

The LEDs can be spaced apart on board 26, with consideration as to their light output and emission wavelength, such that the assembly emits a light illuminance adequate for light therapy. To generate this level of illumination, the assembly generally can include between about 10 and 150 LEDs. Depending on the output of the LEDs, in one embodiment, 24 to 72 LEDs can be used in a device and in another embodiment, 36 to 60 LEDs can be used.

To reduce treatment duration regimens, the LED light can have optimized wavelength emissions with peaks ranging between 400 to 600 nm. In one embodiment, a device emits light with peaks in the 450 to 550 nm range. In another embodiment, a peak wavelength can be in the blue region of the spectrum, which is 420 to 505 nm. Using a light therapy device with light illuminances of less than 2,500 lux and wavelengths peaked in the blue to green region of the spectrum, treatments of acceptable duration can be administered. As an example, treatments for inducing neurogenesis can be completed in ¼ to 4 hours and in most cases, ¼ to 2 hours.

The light generated by the device can be predominantly in the blue to green region such that the emitted light appears distinctly blue/green to a user. However, to enhance acceptance and to reduce the occurrence of problematic after-images, the light can include a range of wavelengths such that the emitted light appears white, but can include a maximum peak in the 400 to 600 nm range.

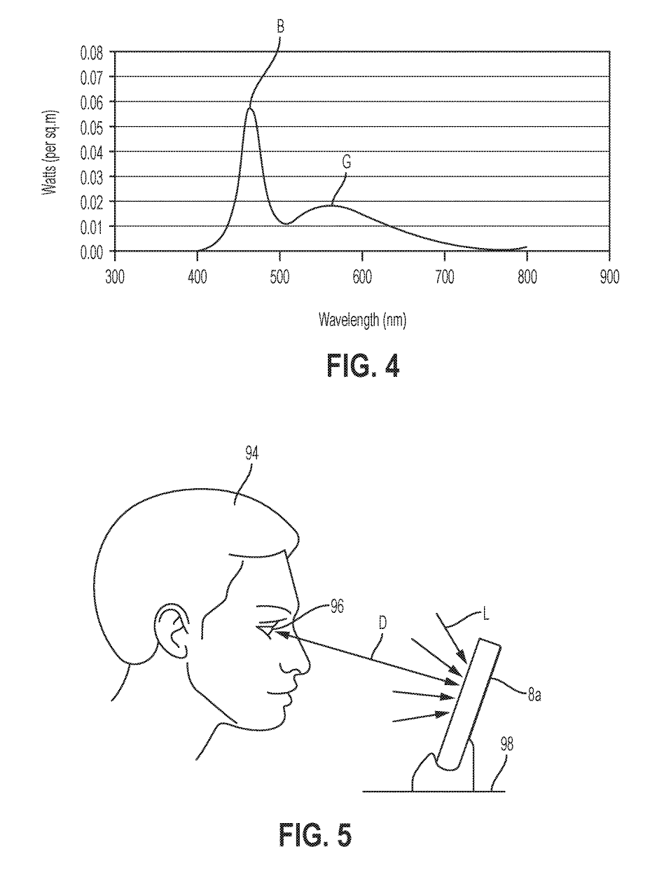

FIG. 4 shows a spectra analysis of light generated by a light therapy device at 12 inches. The light appears as a bright white light, but can have a maximum peak B in the blue wavelengths, between about 446 nm and 477 nm with peak B centered at about 464 nm and with an energy of about 0.055 watts/m². The light emission further can include a secondary but significant peak G in the green wavelengths, between about 505 nm to 600 nm with the greatest output in this peak at about 555 nm. Light emitted can have a maximum peak wavelength in the relevant wavelengths with an energy greater than or equal to 0.01 watts/m². In another embodiment, the emitted light can have a maximum peak has an energy greater than or equal to 0.025 watts/m².

In one embodiment, a light therapy device can emit light wherein of the total light energy emitted at least 25% thereof is of the wavelengths 446 to 477 nm. In another embodiment of a light therapy device, the total light energy emitted is 25 to 40% in the wavelengths 446 to 477 nm.

To achieve a light emission of less than 2,500 lux with peak emissions in the 400 to 600 nm region of the spectrum, various approaches can be taken. In one embodiment, a screen can be used that filters out all or a portion of the less desirable wavelengths. In another embodiment, LEDs capable of emitting only selected wavelengths, for example, including blue, yellow and green, can be used. In yet another embodiment, white light LEDs having selected peak wavelengths can be used.

Device 108 can accommodate a controller 115 to control operation of the device. The controller can include processors, switches, timers, communication functionalities, etc. The controller can turn the light on or off according to presets, stored information or user selections. The controller can calculate a suitable light treatment regime based on installed programs or an input of information. A communication hardware and software can be provided for download of information from external sources such as from the Internet. The controller can include a feature that turns the device on at a pre-set time for and/or off after a specific duration. In one embodiment, the controller controls a switch for the light emitting assembly.

Switches, selectors and/or a touch screen control option can be incorporated to facilitate use. In one embodiment, a device can accommodate a display 82 and a key pad 84.

A speaker 88 can be provided for emitting audible instructions to the user. As an example, the speaker can function to emit an audible signal, such as an alarm, to alert a user to commence or modify a treatment.

If desired, to enhance the usefulness of the device, the calculator can also be programmed with other information including a clock, a standard mathematical calculator or other information such as an address book, etc.

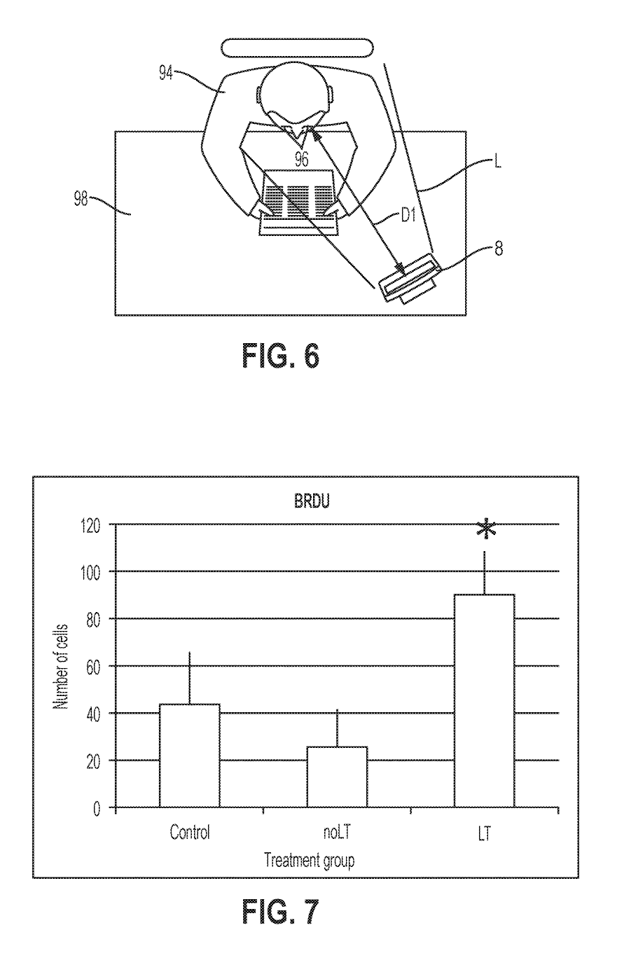

Referring to FIGS. 5 and 6, a method for light therapy to induce neurogenesis can include spacing a light therapy device 8, 8a a distance D, D₁ of 12 or more inches from a user 94. The device can then be operated to emit light L. The light may be at an intensity level of less than 12,000 lux and possibly, less than 2,500 lux as discussed hereinabove, with a maximum peak emission in the 400 to 600 nm region of the spectrum and directing the light toward the eyes 96 of the user. To effect treatment, the light emitting assembly can be directed toward the user, with the emitted light from the device shining into the user's eyes. The present device can be used to provide ocular treatment for all applications and indications and therefore can be used while the users eyes remain substantially open, rather than while they are sleeping.

Typically, the user can position the light emitting assembly of the device between 12-24 inches from their eyes so that a broad beam of light, about shoulder width, impinges on the user. The treatment field generated by the device can offer personal light therapy. Since the treatment field at normal spacings can be shoulder width, the device can be used without shining the emitted light onto adjacent persons.

The device can be situated on a support surface 98 such as a table, desk, holder, etc. or supported in other ways, so as to emit light upwards towards the user's eyes. The device can be offset, for example, 30 to 45°, from a position directly in front of the user, so that the light shines directly on the periphery of the retina (outside the fovea), which is thought to be the location of some photoreceptors of interest.

The user's eyes should be open to effect treatment, although blinking to a normal degree is expected and acceptable. It is not necessary for the user to stare directly into the light from the device. Indeed, the light is generally sufficiently bright so that the user instinctively knows not to do so.

Treatment times for inducing neurogenesis are typically 15-60 minutes/day. In one embodiment, the treatment may take place in the first 6 hours after waking (i.e. in the morning), for example, be undertaken be as soon as possible upon waking. The treatments may be administered regularly for example on a daily basis for a treatment period or until an acceptable result is observed.

### EXAMPLES

The effect of LED light treatment on neurogenesis was tested using adult rats. It is well known that rat models are good indicators for human neuroresponse.

### Example 1

In one experiment, the hypothesis that LED light therapy, after circadian disruption, can increase adult neurogenesis in the hippocampus was tested. Some of the benefit from LED light therapy could be related to improvement of functioning of some parts of the cerebral cortex, including the hippocampus which is critically involved in forming new memories.

Animals

Adult male Long Evans rats were obtained from Charles River Laboratory Animal Supply Company located in Quebec. These rats were not transfected with any genes relating to light response. All of the animals arrived to the Canadian Centre for Behavioural Neuroscience (CCBN) under the Protocol #1004 Approved by the University of Lethbridge Animal Welfare Committee. All behavioral testing took place at the University of Lethbridge Canadian Centre for Behavioural Neuroscience.

Upon arrival, rats were singly housed in Plexiglas hanging tubs with ground corncob bedding. All rats had free access to food and water, and were maintained on a 12:12 light/dark cycle during the acclimation period. Rats weighed 300-350 gm at the beginning of the experiment. Each animal was tail marked with a unique identifier for clear identification of the animal. All rats had access to environmental enrichment and running wheels.

Following 14 days of acclimation rats were randomly assigned to one of four treatment groups using a random number generator. The treatment groups were: 1) Control—no circadian disruption and no ocular light treatment, 2) Group 2—circadian disruption and no ocular light treatment, 3) Group 3—circadian disruption and no ocular light treatment.

The cages for Group 1 were in a room with regular (non-LED) room lights and the room lights were maintained at a set 12:12 light:dark cycle. The cages for Groups 2 and 3 were in rooms with regular (non-LED) room lights, but where the room lights were deviated from a set 12:12 light: dark cycle.

Lights for Ocular Light Therapy

Litebook Elite™ lights available from the current applicant were employed for the tests. Each light included a 10×15 cm screen emitting white light from 24 white light LEDs. The light emits less than 2500 lux at 12 inches and has a spectrum similar to that shown in FIG. 4.

Each light was adapted to hang on the outside of a clear Plexiglas cage. The lights were present on the cages for the duration of the experiment, but only connected to power for the treatment period. During the week of treatment, the lights were connected to power to deliver the timed exposure.

It was confirmed that the spectral output was the same outside of a Plexiglas cage as inside, indicating that there was no filtering effect as the light passed through the Plexiglas.

Blackout curtains were used to ensure that only rats in the appropriate groups were exposed to the light treatment.

Circadian Disruption

Chronic circadian disruption was achieved using a well-tested procedure with rats (see Table I).

TABLE I
Schedule of phase shifting to cause circadian disruption

| Day | Light schedule |
|---|---|
| 1 | Lights off at 16:30 |
| 2 | Lights off at 13:30 |
| 3 | Lights off at 10:30 |
| 4 | Lights off at 7:30 |
| 5 | Lights off at 4:30 |
| 6 | Lights off at 1:30 |
| 7-16 | Re-entrainment - lights off at 22:30 |
| 17 | Lights off at 19:30 |
| 18 | Lights off at 16:30 |
| 19 | Lights off at 13:30 |
| 20 | Lights off at 10:30 |
| 21 | Lights off at 7:30 |
| 22 | Lights off at 4:30 |
| 23-32 | Lights off at 1:30 |

Therapeutic intervention took place during the re-entrainment phase after circadian disruption. All physiological measurements took place following six days of ocular light therapy.

Method

To quantify adult hippocampal neurogenesis we used three immunolabeling methods with 10 rats in each group. First, we administered bromodeoxyuridine (BrdU) on the final day of circadian disruption (120 mg/kg, i.p.). BrdU is taken up by cells that are actively synthesizing new DNA and is permanently incorporated into nuclear DNA of daughter cells. Rats were euthanized seven days after BrdU administration. Second, we labeled tissue with an antibody to Ki67, a protein expressed in cells that are actively cycling at the time of euthanasia. Third, we labeled tissue with an antibody to doublecortin (DCX) a protein only expressed in immature neurons. Using the combination of these techniques we can determine the number of cells born just before LED light therapy (or no therapy) that survive for one week, the number of cells that are actively cycling at the end of therapy and the number of new neurons born during the week of therapy.

Primary antibodies were as follows: rat anti-BrdU (BU1/75, product # OBT0030, Oxford Biotechnology, Oxfordshire, UK); goat anti-DCX (product #sc-8066, Santa Cruz Biotechnology, Santa Cruz, Calif.); rabbit anti-Ki-67 (product #NCL-Ki-67p, Novocastra Ltd., Newcastle Upon Tyne, UK.

Secondary antibodies were as follows: Alexa Fluor 488 chicken anti-rat (product #A21470, Molecular Probes, Eugene, Ore.); biotin-SP-conjugated donkey anti-goat (product #705-065-147; Jackson ImmunoResearch, West Grove, Pa.); Alexa Fluor 488 donkey anti-rabbit (product #A21206, Molecular Probes).

Perfusions, Histology, and Immunohistochemistry

After a lethal injection of sodium pentobarbital (150 mg/ml), animals were transcardially perfused with 150 ml of 0.1 M phosphate-buffered saline (PBS), pH 7.4, followed by 200 ml of 4% paraformaldehyde in 0.1 M PBS. Brains were removed and post-fixed in 4% paraformaldehyde in PBS for 24 hours at 4° C. This solution was then replaced by 30% sucrose in PBS containing 0.02% sodium azide and, when the brains sunk, they were cut at 40 iam into either a ⅙ section sampling fraction on a freezing sliding microtome (American Optical, model #860; Buffalo, N.Y.). With each brain, the collection of coronal sections started at a random point before the beginning of the dentate gyms, and was sectioned exhaustively through its entire rostral-caudal axis. Sections were collected into PBS containing 0.02% sodium azide and stored at 4° C. until processed.

Immunohistochemistry was conducted as free-floating sections, using 0.1 M PBS with 0.3% Triton X-100 as a diluent in all cases. Incubation times were 24 hours for all primary antibodies and secondary antibodies, and 1 hour for tertiary reagents. Incubations were carried out at room temperature on a rotating table.

To determine the number of new cells and immature neurons within the hippocampus, two series from each animal were labeled, one with rabbit anti-Ki-67 (1:1,000) and the other goat anti-DCX (1:500), using Alexa-488-conjugated donkey anti-rabbit (1:250) and a biotinylated donkey anti-goat (1:6,000) antibodies as secondary reagents; DAPI was used as a counterstain to delineate the granule cell layer. Streptavidin-conjugated Alexa 568 (1:500) was subsequently used to detect DCX.

To detect the presence of BrdU, the tissue was processed through several DNA denaturing steps in order to retrieve the BrdU epitope. Briefly, the tissue was first exposed to a solution of 2× saline sodium citrate buffer in 50% formamide at 65° C., followed by two rinses in 2× saline sodium citrate buffer alone at room temperature. Sections were then placed into 2N HCl at 37° C. for 30 minutes. After several rinses in PBS over approximately 1.5 hours, the tissue was then placed into rat anti-BrdU (1:100) and goat anti-DCX (1:500) primaries. Following primary incubations, the tissue was rinsed three times in PBS, and placed into Alexa Fluor 488 chicken anti-rat (1:600) and biotin-conjugated donkey anti-goat (1:6,000). Sections were rinsed again, and placed into streptavidin-conjugated Alexa 568 (1:500) before mounting. Sections were mounted out of PBS and coverslipped with a glycerol-based antifade reagent (9.8% polyvinyl alcohol, 2.5% 1,4-diazabicyclo [2.2.2]octane, 24% glycerol in 0.1M Tris-HCl, pH 8.3; all obtained from Sigma). Signals were subsequently analyzed under appropriate filters using a Zeiss Axioskop2 MotPlus microscope or a Nikon C1 confocal microscope where appropriate. Control experiments included the incubation of sections in the absence of primary antibodies. All images were captured using a QImaging Retiga EXi CCD camera (Burnaby, British Columbia).

Unbiased stereological estimates of the number of cFos-positive cells were made using the optical fractionator method in StereoInvestigator (9.03 32-bit; MBT Bioscience-MicroBrightfield, Inc., Williston, Vt., USA). Labeled cells were counted using a 80×80 counting matrix and a 40× objective through the dorsal (septal) half of the hippocampal dentate gyms in both hemispheres. Statistical analyses were conducted using MS Excel for Mac version 14.4.1 with significance level p<0.05 and with one-tail since a priori we are testing if LED light therapy enhances neurogenesis.

Results and Discussion.

BrdU. FIG. 7 shows the results of counting BrdU-positive cells in the hippocampus of the rats in each treatment group. There was a non significant trend for the Control group to have more BrdU positive cells than the no light therapy (LT) group (p<0.06). In contrast, the LT group showed more than twice the number of new cells than the rats of the other groups. This difference is significant at the p<0.001 level.

The rats in the group receiving LED light treatment had significantly more BrdU positive cells than the Control+no LT groups (p<0.001). There was a non significant trend for the rats of the no LT group to have fewer BrdU positive cells than those in the Control group.

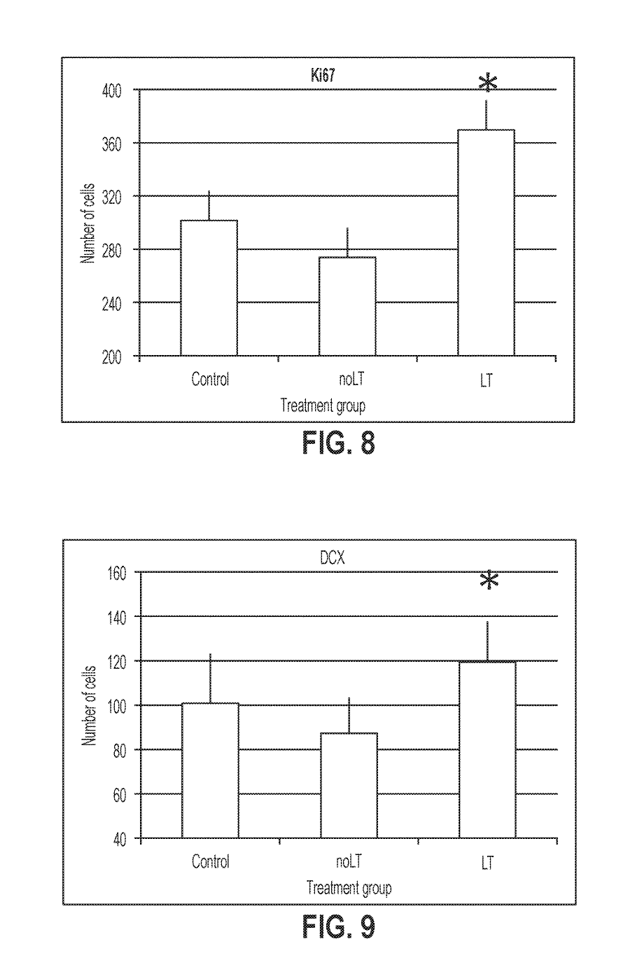

Ki67. FIG. 8 shows the results of counting Ki67 positive cells in the hippocampus. There was a non significant trend for the Control group to have more Ki67 positive cells than the no LT group (p=0.16). The rats receiving LED light treatment showed significantly more labeled cells than the Control+no LT groups (p<0.0006).

The rats in the group receiving LED light treatment had significantly more Ki67 positive cells than the Control+no LT groups (p<0.0006). There was a non significant trend for the rats of the no LT group to have fewer Ki67 positive cells than those in the Control group.

DCX. FIG. 9 shows the results of counting the DCX positive cells in the hippocampus. Similar to the other immunolabeling results, there was a non significant trend for the Control group to have more DCX positive cells than the no LT group (p=0.13). The group with LED light treatment showed significantly more DCX positive cells than the other two groups (p<0.03).

The rats in the group receiving LED light treatment had significantly more DCX positive cells than the Control+no LT groups (p<0.03). There was a non significant trend for the rats of the no LT group to have fewer DCX positive cells than those in the Control group.

The outcome of the evaluation of adult neurogenesis in the hippocampus was unequivocal on all three of the measures. LED light treatment enhances neurogenesis. Adult neurogenesis involves several processes, creation of new cells (proliferation or cell division), maturation (differentiation in to adult neurons) and many factors that influence survival of daughter cells. The fact that the number of DCX cells was increased by treatment conclusively means that more new neurons are generated by LED light treatment. In addition, the fact that the number of Ki67 cells was also increased means that LED light treatment increases the pool of cycling cells in the hippocampus, that is, the rate of cell division or proliferation rate is increased. Finally, our observation that there are more BrdU positive cells implies that LED light treatment improves the brain environment to favour cell survival. This inference is supported by considering that the BrdU was administered prior to onset of treatment, ruling out proliferation as the cause of increased number of BrdU labeled cells.

Although our tests shown that LED light therapy can enhance neurogenesis under the conditions of the present experiment, the tests do not show to what extent this effect will generalize to healthy subjects who have not experienced circadian disruption or who are not engaging in vigorous, daily voluntary exercise.

### General Discussion

It is well established that some aspects of long-term spatial memory depends upon the health of the hippocampus. Importantly, when we measured the impact of LED light treatment on adult neurogenesis in the hippocampus, we found evidence that proliferation and survival of new neurons may actually be enhanced relative to the normal control participants. LED light treatment may also be applicable to humans seeking cognitive benefit.

It is important to note that adult neurogenesis declines markedly with age and is correlated with age-related memory decline. One should consider that the benefit of LED light treatment we establish here on adult neurogenesis is in the context of a rat model that experienced chronic circadian disruption and daily vigorous voluntary exercise. The extent to which the LED light neurogenesis effect is dependent on or modulated by these two factors is an open question. The answer to this question may indicate that LED light could have a physiological and cognitive benefit that is much broader than reversing circadian disruptions.

Conclusions Significant benefits of a week of daily LED light treatment were established in adult neurogenesis in a rat model of chronic circadian disruption. These represent encouraging outcomes with direct implications for benefit in relevant human conditions and for possible brain mechanisms that directly influence age-related and other cognitive processes.

### Example 2

Animals

Adult male Long Evans rats, as noted above, were obtained from Charles River Laboratory Animal Supply Company located in Quebec. All of the animals arrived to the Canadian Centre for Behavioural Neuroscience (CCBN) under the Protocol #1004 Approved by the University of Lethbridge Animal Welfare Committee. All behavioral testing took place at the University of Lethbridge Canadian Centre for Behavioural Neuroscience.

Upon arrival, rats were singly housed in Plexiglas hanging tubs with ground corncob bedding. All rats had free access to food and water, and were maintained on a 12:12 light/dark cycle during the acclimation period. Rats weighed 300-350 gm at the beginning of the experiment. Each animal was tail marked with a unique identifier for clear identification of the animal.

Following 14 days of acclimation rats were randomly assigned to one of four treatment groups using a random number generator. The treatment groups were: 1) Control—no wheel running and no ocular light treatment, 2) Group 2—no wheel running and ocular light treatment, 3) Group 3 wheel running and no ocular light treatment.

In all groups, the cages were in rooms with regular (non-LED) room lights and the room lights were maintained at a set 12:12 light:dark cycle.

Rats in the exercise groups were allowed continuous access to running wheels (exercise). Running wheels were not placed into the cages of the groups with no exercise.

Lights for Ocular Light Therapy

Litebook Elite™ lights available from the current applicant were employed for the tests. Each light included a 10×15 cm screen emitting white light from 24 white light LEDs. The light emits less than 2500 lux at 12 inches and has a spectrum similar to that shown in FIG. 4.

Each light was adapted to hang on the outside of a clear Plexiglas cage. They were automatically controlled to turn on and off via a single timer unit. Blackout curtains were used to ensure that only rats in the appropriate groups were exposed to the light treatment.

Experimental Procedure

On day 14, after acclimation, each rat was given an injection of BRDU. Light therapy commenced on the morning of day 15 for the rats in the light therapy groups. Specifically, beginning on Day 15 rats in the light treatment groups received 30 min of LED light exposure beginning at the time of room lights-on for seven consecutive days. For the same seven days rats in the exercise groups had continuous access to running wheels.

On the 22^{nd} day all rats were euthanized and their brains processed for measuring neurogenesis. To quantify adult hippocampal neurogenesis we used three immunolabeling methods with 12 rats in each group using BrdU, Ki67, and doublecortin antibodies. Several of the brains were not usable after histological processing.

As noted, bromodeoxyuridine (BrdU) was administered on Day 14 before initiating treatments (120 mg/kg, i.p.). BrdU is taken up by cells that are actively synthesizing new DNA and is permanently incorporated into nuclear DNA of the daughter cells.

After euthanization, tissue was labeled with an antibody to Ki67, a protein expressed in cells that are actively cycling at the time of euthanasia. Tissue was also labeled with an antibody to doublecortin (DCX), a protein only expressed in immature neurons.

Using the combination of these techniques it is possible to determine the number of cells born just before LED light treatment (or no treatment) that survive for one week (BrdU), the number of cells that are actively cycling at the end of treatment (Ki67) and the number of new neurons born during the week of treatment (DCX).

Primary antibodies were as follows: rat anti-BrdU (BU1/75, product # OBT0030, Oxford Biotechnology, Oxfordshire, UK); goat anti-DCX (product #sc-8066, Santa Cruz Biotechnology, Santa Cruz, Calif.); and rabbit anti-Ki-67 (product #NCL-Ki-67p, Novocastra Ltd., Newcastle Upon Tyne, UK.

Secondary antibodies were as follows: Alexa Fluor 488 chicken anti-rat (product #A21470, Molecular Probes, Eugene, Ore.); biotin-SP-conjugated donkey anti-goat (product #705-065-147; Jackson ImmunoResearch, West Grove, Pa.); and Alexa Fluor 488 donkey anti-rabbit (product #A21206, Molecular Probes).

Perfusions, Histology, and Immunohistochemistry

After a lethal injection of sodium pentobarbital (150 mg/ml), animals were transcardially perfused with 150 ml of 0.1 M phosphate-buffered saline (PBS), pH 7.4, followed by 200 ml of 4% paraformaldehyde in 0.1 M PBS. Brains were removed and post-fixed in 4% paraformaldehyde in PBS for 24 hours at 4° C. This solution was then replaced by 30% sucrose in PBS containing 0.02% sodium azide and, when the brains sunk, they were cut at 40 μm into either a ⅙ section sampling fraction on a freezing sliding microtome (American Optical, model #860; Buffalo, N.Y.). With each brain, the collection of coronal sections started at a random point before the beginning of the dentate gyms, and was sectioned exhaustively through its entire rostral-caudal axis. Sections were collected into PBS containing 0.02% sodium azide and stored at 4° C. until processed.

Immunohistochemistry was conducted as free-floating sections, using 0.1 M PBS with 0.3% Triton X-100 as a diluent in all cases. Incubation times were 24 hours for all primary antibodies and secondary antibodies, and 1 hour for tertiary reagents. Incubations were carried out at room temperature on a rotating table.

To determine the number of new cells and immature neurons within the hippocampus, two series from each animal were labeled, one with rabbit anti-Ki-67 (1:1,000) and the other goat anti-DCX (1:500), using Alexa-488-conjugated donkey anti-rabbit (1:250) and a biotinylated donkey anti-goat (1:6,000) antibodies as secondary reagents; DAPI was used as a counterstain to delineate the granule cell layer. Streptavidin-conjugated Alexa 568 (1:500) was subsequently used to detect DCX.

To detect the presence of BrdU, the tissue was processed through several DNA denaturing steps in order to retrieve the BrdU epitope. Briefly, the tissue was first exposed to a solution of 2× saline sodium citrate buffer in 50% formamide at 65° C., followed by two rinses in 2× saline sodium citrate buffer alone at room temperature. Sections were then placed into 2N HCl at 37° C. for 30 minutes. After several rinses in PBS over approximately 1.5 hours, the tissue was then placed into rat anti-BrdU (1:100) and goat anti-DCX (1:500) primaries. Following primary incubations, the tissue was rinsed three times in PBS, and placed into Alexa Fluor 488 chicken anti-rat (1:600) and biotin-conjugated donkey anti-goat (1:6,000). Sections were rinsed again, and placed into streptavidin-conjugated Alexa 568 (1:500) before mounting.

Sections were mounted out of PBS and cover-slipped with a glycerol-based antifade reagent (9.8% polyvinyl alcohol, 2.5% 1,4-diazabicyclo[2.2.2]octane, 24% glycerol in 0.1M Tris-HCl, pH 8.3; all obtained from Sigma). Signals were subsequently analyzed under appropriate filters using a Zeiss Axioskop2 MotPlus microscope or a Nikon C 1 confocal microscope where appropriate. Control experiments included the incubation of sections in the absence of primary antibodies. All images were captured using a QImaging Retiga EXi CCD camera (Burnaby, British Columbia).

Unbiased stereological estimates of the number of cFos-positive cells were made using the optical fractionator method in StereoInvestigator (9.03 32-bit; MBT Bioscience-MicroBrightfield, Inc., Williston, Vt., USA). Labeled cells were counted using a 80×80 counting matrix and a 40× objective through the dorsal (septal) half of the hippocampal dentate gyms in both hemispheres. Statistical analyses were conducted using MS Excel for Mac version 14.4.1 with significance level p<0.05 and with one-tail since a priori we are testing if LED light treatment enhances neurogenesis.

Results and Discussion.

Ki67

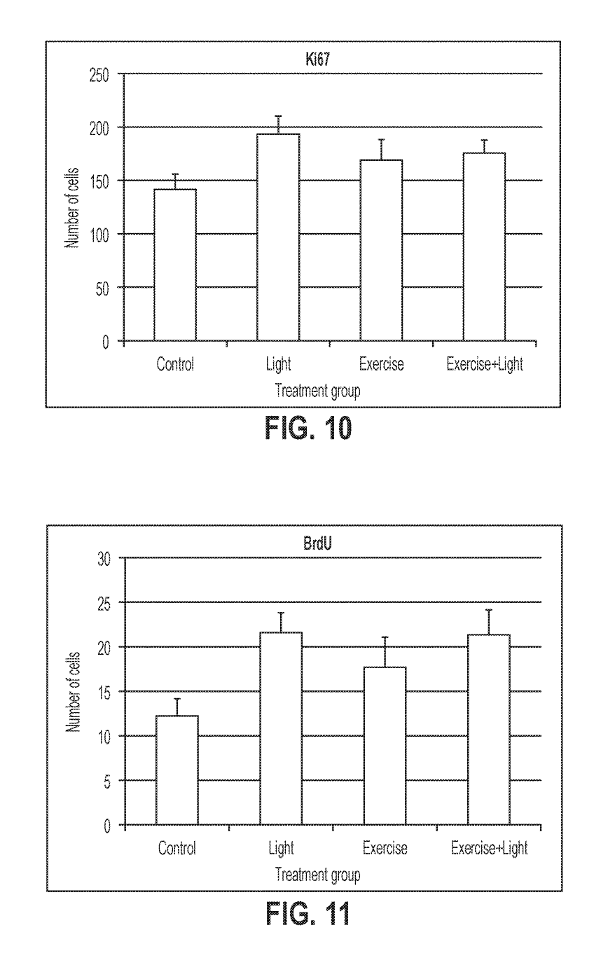

FIG. 10 shows the results of counting Ki67 positive cells in the hippocampus. The rats receiving LED light treatment showed significantly more labeled cells than the Control group (p=0.03). The other treatment groups did not reliably differ from the Control group. The rats in the group receiving LED light treatment had significantly more Ki67 positive cells than the Control rats (p<0.03). There was a non significant trend for the rats of the Exercise+Light and Exercise groups to have more Ki67 positive cells than those in the Control group (p=0.07 and p=0.17 respectively).

BrdU

FIG. 11 shows the results of counting BrdU-positive cells in the hippocampus of the rats in each treatment group. We found significantly more BrdU positive cells in the rats of the Light group than the Control group (p=0.02). In contrast, the Exercise group showed the same trend but it was not statistically significant. The Exercise+Light group was very similar to the Light alone group and they showed significantly more BrdU cells than the Control group (p=0.02). The rats in the group receiving LED light treatment had significantly more BrdU positive cells than the Control group (p=0.02). There was a non significant trend for the rats of the Exercise group to have more BrdU positive cells than those in the Control group and the Exercise+Light group closely resembled the Light group.

DCX

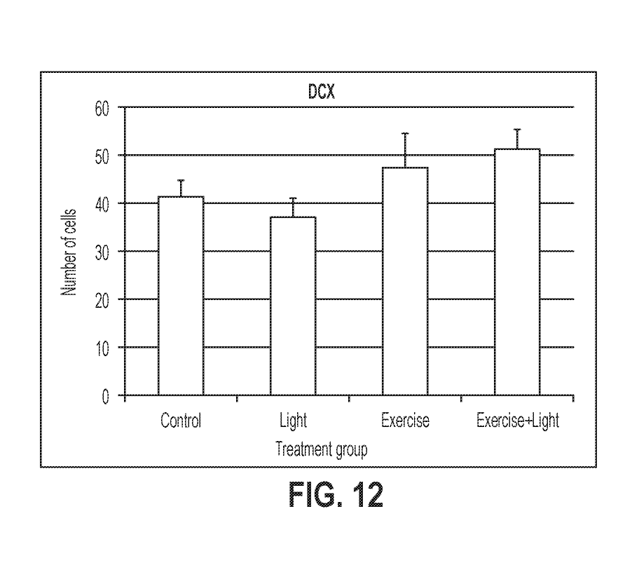

FIG. 12 shows the results of counting the DCX positive cells in the hippocampus. Neither LED light alone nor Exercise alone significantly affected the number of DCX positive cells. The combination of Exercise+Light treatment produced more DCX positive cells than the Control group (p=0.04). In particular, as shown in FIG. 12, the rats in the group receiving LED light treatment were not different that the Control group (p=0.22). Only the Exercise+Light group had significantly more DCX cells than the Control group (p=0.04).

The outcome of the evaluation of adult neurogenesis in the hippocampus produced significant results on all three of the measures. This LED light treatment clearly enhanced hippocampal neurogenesis in the adult rats. Adult neurogenesis involves several processes: creation of new cells (proliferation or cell division), maturation (differentiation in to adult neurons) and many factors that influence survival of daughter cells. The fact that the number of Ki67 and BrdU cells was increased by treatment conclusively means that more new cells are generated by LED light treatment and that LED light treatment increases cell survival. In addition, the fact that the number of DCX cells was increased only when Light and Exercise were combined indicates that both of these treatments have a smaller effect on the number of immature neurons. Since newly born cells take several days before they begin to express DCX, in retrospect a seven-day treatment may have been too short to see a full evolution of the effect of treatments.

It is established that LED light therapy can enhance adult neurogenesis in the absence of any sleep disturbance or circadian disruption. On two out of three of the measures of adult neurogenesis the beneficial effect of LED light therapy was clear even in sedentary animals. The third measure indicated a significant effect only in exercising animals but the time of exposure to treatments may have been too short to see a full evolution of the treatments.

Numerous modifications, variations and adaptations may be made to the particular embodiments described above without departing from the scope of the invention as defined in the claims.

What is claimed is:

1. A method for inducing neurogenesis in the brain of a mammal comprising administering to the mammal a light treatment from an LED light source, wherein the treatment induces neurogenesis.

2. The method of claim 1, wherein administering includes operating a device to emit light toward and shining into the mammal's eyes.

3. The method of claim 1, wherein the light treatment includes administering white light from the LED light source.

4. The method of claim 1, wherein administering the light treatment comprises light with a maximum peak in the 400 nm to 600 nm range of the light spectrum.

5. The method of claim 1, wherein administering the light treatment comprises light with a maximum peak between about 420 nm and 505 nm.

6. The method of claim 1, wherein administering the light treatment comprises light with at least 25% of the wavelengths 446 nm to 477 nm.

7. The method of claim 1, wherein the LED light source emits white light.

8. The method of claim 1, wherein administering the light treatment comprises light at an intensity of between 500 and 12,000 lux at 12 inches.

9. The method of claim 1, said method further comprising exercise.

10. The method of claim 1, wherein the LED light source is a device comprising (i) an outer housing, and (ii) a light emitting assembly in the housing and operable to emit light from the device, the light emitting assembly including a plurality of LEDs capable of generating an output of light of between 500 and 12,000 lux at 12 inches.

11. A method of treating a neurodegenerative condition in a mammal, comprising administering to the mammal a light treatment from an LED light source, wherein the light treatment treats the neurodegenerative condition.

12. The method of claim 1, wherein the neurogenesis occurs in the hippocampal region of the brain.

13. The method of claim 11, wherein the treatment of the neurodegenerative disorder results in neurogenesis in the hippocampal region of the brain.

14. The method of claim 11, wherein the light treatment comprises operating the LED light source to emit light toward and shining into the mammal's eyes.

15. The method of claim 14, wherein the LED light source emits white light.

16. The method of claim 11, wherein the LED light source has a maximum peak in the 400 nm to 600 nm range of the light spectrum.

17. The method of claim 11, wherein the LED light source has a maximum peak between about 420 nm and 505 nm.

18. The method of claim 11, wherein at least 25% of the light is in wavelengths 446 nm to 477 nm.

19. The method of claim 11, wherein the treatment occurs 6 hours after waking.

20. The method of claim 11, wherein the LED light source has an intensity of between 500 and 12,000 lux at 12 inches.

21. The method of claim 11, said method further comprising exercise.

22. The method of claim 11, wherein the LED light source is a device including (i) an outer housing, and (ii) a light emitting assembly in the housing and operable to emit light from the device, the light emitting assembly including a plurality of LEDs capable of generating capable of generating an output of light of between 500 and 12,000 lux at 12 inches.
